(19) United States
(12) Patent Application Publication
(10) Pub. No.: US 2026/0060609 A1
(43) Pub. Date: Mar. 5, 2026

(54) Device for determining insomnia

(71) Applicant: SOMNOmedics AG, Randersacker (DE)

(72) Inventor: Gert KÜCHLER, Randersacker (DE)

(21) Appl. No.: 19/317,634

(22) Filed: Sep. 3, 2025

(30) Foreign Application Priority Data
Sep. 3, 2024 (DE) 10 2024 208 368.2

(51) Int. Cl. A61B 5/00; A61B 5/291; A61B 5/374; G16H 40/63

(52) U.S. Cl. CPC A61B 5/4815; A61B 5/374; A61B 5/7203; G16H 40/63; A61B 5/291; A61B 2505/07

(57) ABSTRACT

The device (1) is used to determine insomnia in a patient (8). It has a sensor unit (2) for recording at least one EEG measurement signal of a brain wave of the patient (8) and a control and evaluation unit (3) for evaluating the recorded EEG measurement signal. The control and evaluation unit (3) is configured to carry out an objective assessment routine and in doing so, for the subsequent recording of the EEG measurement signal, to give the patient (8) a stipulation to open his eyes for the duration of an eyes-open period and to close his eyes for the duration of an eyes-closed period, and then to initiate the recording of the EEG measurement signal during at least one eyes-open period and during at least one eyes-closed period. The control and evaluation unit (3) is further configured to perform an evaluation of the EEG measurement signal thus recorded and in doing so to extract from the EEG measurement signal at least one first partial measurement signal from the at least one eyes-open period and at least one second partial measurement signal from the at least one eyes-closed period, and to decide on the presence of insomnia if the at least one second partial measurement signal from the at least one eyes-closed period has a lower proportion of α waves than in a healthy individual.

The contents of German patent application DE 10 2024 208 368.2 is incorporated by reference.

The invention relates to a device for determining insomnia in a patient.

Insomnia refers to difficulties in falling asleep and/or staying asleep. It is one of the most widespread sleep disorders, affecting up to 30% of the adult population, and often goes unrecognised. Despite the high prevalence of this sleep disorder, there is currently no suitable screening method or device for ascertaining an objective parameter with which to diagnose insomnia. Even with polysomnography, which is very complex and expensive and can only be performed in a sleep laboratory, it has not yet been possible to determine an objective insomnia parameter of this type.

Instead, for some time now, an insomnia diagnosis has been made solely on the basis of the subjective state of an affected individual. Sleep questionnaires, which the affected individual completes the following morning after his sleeping hours at night, form the sole basis for the insomnia diagnosis. The findings thus recorded using the sleep questionnaires are highly subjective and depend, at least in part, on the pattern and quality of the previous sleep.

DE 20 2022 106 837 U1 discloses a sleep diagnosis device suitable for use in the patient's home environment. Electrodes applied by the patient to his own scalp are used to record electrophysiological signals from the patient, which are then transmitted to a multi-part or multi-component control/evaluation unit for analysis. These electrophysiological signals are in particular electroencephalography (EEG) signals for recording the electrical activity of the brain, electrooculography (EOG) signals for recording eye movements, and/or electromyography (EMG) signals for recording muscle activity in the head area. Although this device is used for sleep diagnosis, it does not make it possible to diagnose insomnia.

The object of the invention is to provide a device of the type indicated at the outset having improved properties by comparison with the prior art.

To achieve this object, a device according to the features of claim 1 is set out. The device according to the invention comprises a sensor unit, at least for recording an EEG measurement signal of a brain wave of the patient, as well as a control and evaluation unit for evaluating the recorded EEG measurement signal. The control and evaluation unit is configured to carry out an objective assessment routine and in doing so, for the subsequent recording of the EEG measurement signal, to give the patient a stipulation to open the eyes for the duration of an eyes-open period and to close the eyes for the duration of an eyes-closed period, and then to initiate the recording of the EEG measurement signal during at least one eyes-open period and during at least one eyes-closed period. The control and evaluation unit is further configured to perform an evaluation of the EEG measurement signal thus recorded and in doing so to extract from the EEG measurement signal at least one first partial measurement signal from the at least one eyes-open period and at least one second partial measurement signal from the at least one eyes-closed period, and to decide on the presence of insomnia if the at least one second partial measurement signal from the at least one eyes-closed period has a lower proportion of α waves than in a healthy individual.

The abbreviation “EEG” stands for electroencephalography or electroencephalogram.

During the objective assessment routine, in particular at least one eyes-open period and at least one eyes-closed period are provided. The eyes of the patient or the user of the insomnia determination device are open during the eyes-open period and closed during the eyes-closed period.

A healthy individual normally has high brain activity or high neuronal activity when awake with his eyes open, resulting in the recorded EEG signal containing a significant proportion of what are known as β waves. If the visual stimuli are removed, for example by closing the eyes, neuronal activity decreases and synchronised neuronal discharges occur. The recorded EEG signal then contains a significant proportion of what are known as α waves, which have a reduced frequency by comparison with the β waves. The α waves occur primarily when the eyes are closed and the individual is relaxed and awake, and are replaced by β waves when the eyes are opened, and vice versa. This phenomenon is known as the Berger effect.

It has been found that, upon closing their eyes, insomniacs do not experience a shift to α waves comparable to that in healthy individuals, this being a possible correlate of a reduced ability to relax. Therefore, the absence of α waves upon closing the eyes can be used as a measure of the ability to relax. The device according to the invention uses this finding as a basis for deciding whether insomnia is present. This decision is preferably automated and in particular also objective, being based on the EEG measurement signal recorded and subsequently evaluated with the eyes open and closed as part of the objective assessment routines.

One of the difficulties with conventional insomnia diagnosis based on sleep questionnaires is the conflicting assessment of sleep pressure and current vigilance (=alertness). If sleep pressure is high and vigilance is low, there will be no difficulty in falling asleep. If both sleep pressure and vigilance are high, this can lead to difficulty in falling asleep. If sleep pressure is high and vigilance is merely increased, the ability to fall asleep may still be good, but after the first deep sleep phase, once sleep pressure is reduced, ability to stay asleep may deteriorate. All of this may be taken into account when the sleep questionnaire is completed the next morning, and can distort an insomnia diagnosis made on this basis.

This difficulty is avoided by the insomnia determination device, since the insomnia diagnosis is based on the absence of a significant proportion of α waves in the EEG measurement signal with closed eyes, which has been recognised as an objective criterion. This objective criterion has a very good correlation with the patient's subjective state of insomnia according to the sleep questionnaires he completes. Overall, the device according to the invention greatly simplifies and improves insomnia diagnosis, and also makes it more objective.

In particular, the device may have additional functions in addition to determining insomnia, such as advantageously simple and cost-effective detection or recording of the sleep stages of the patient, who in this case is monitored during sleep, in particular by the device. In this favourable embodiment, the device can thus perform sleep screening. In this case, it may also be referred to as a screening device and, in this favourable embodiment, it is in particular multifunctional.

As well as the EEG measurement signal for recording the electrical activity of the brain, the sensor unit is in particular configured to record further electrophysiological measurement signals, such as an electrooculography (EOG) measurement signal for recording eye movement or an electromyography (EMG) measurement signal for recording muscle activity in the head area. In addition, the sensor unit is preferably configured to record further measurement signals, such as a body position measurement signal and an acoustic measurement signal of a noise caused by the patient during sleep, in particular a snoring noise. In addition, the sensor unit may also be configured to additionally record at least one measurement signal independent of the patient, such as a light measurement signal, in particular that of the ambient light. These further measurement signals are then used in particular to detect different sleep states or sleep stages of the patient.

Advantageous embodiments of the device will be apparent from the features of the claims dependent on claim 1.

In one favourable embodiment, the control and evaluation unit is configured to check the at least one second partial measurement signal for the presence of a periods in which α waves occur, and to ascertain an α period duration for each detected a period, and to decide on the presence of moderate to severe insomnia if the sum of all α period durations is less than 20% of a total duration of the at least one second partial measurement signal. Preferably, the α waves within the α periods are dominant over other wave components of the at least one second partial measurement signal. In particular, the α waves are predominant, preferably significantly so, by comparison with other wave components of the at least one second partial measurement signal.

In a further favourable embodiment, the control and evaluation unit is configured to perform a comparison between a first frequency content of the at least one first partial measurement signal from the at least one eyes-open period and a second frequency content of the at least one second partial measurement signal from the at least one eyes-closed period, and, for the comparison, to determine, from the first frequency content of the at least one first partial measurement signal from the at least one eyes-open period, a first α characteristic for an α frequency range and a first β characteristic for a β frequency range as well as a first α/β ratio as the quotient of the first α characteristic over the first β characteristic, to determine, from the second frequency content of the at least one second partial measurement signal from the at least one eyes-closed period, a second α characteristic for the α frequency range and a second β characteristic for the β frequency range as well as a second the α/β ratio as the quotient of the second α characteristic over the second β characteristic, and to determine a relative change between the first and the second α/β ratio. In this favourable embodiment, both the influence of the α waves (described by the first and the second α characteristic) and the influence of the β waves (described by the first and the second β characteristic) are taken into account. The α waves are in the α frequency range, which extends in particular over frequencies between 8 Hz and 14 Hz, preferably between 8 Hz and 13 Hz and preferably between 8 Hz and 12 Hz. The β waves are in the β frequency range, which extends in particular over frequencies between 14 Hz and 32 Hz, preferably between 14 Hz and 30 Hz and preferably between 13 Hz and 30 Hz. Neuronal activity is different in every individual and depends in particular on age and gender, among other things. To minimise or even completely eliminate the influence of these individual differences on the insomnia determination, this favourable embodiment relates the α activity necessary for insomnia determination to the β activity. This results in a standardisation which takes the individual neuronal activity of the patient in question into account, improving the accuracy of the insomnia determination results. It has been found that the relative change between the first α/β ratio with eyes open and the second α/β ratio with eyes closed surprisingly has a very good correlation with the patient's subjective state of insomnia according to the sleep questionnaires he has completed. Furthermore, it has been found that in healthy individuals there is a significant relative change between the first α/β ratio with eyes open and the second α/β ratio with eyes closed, whereas in insomniacs this relative change is only very small if it even occurs at all.

The control and evaluation unit is in particular configured to transform the at least one first partial measurement signal (for open eyes) into the frequency domain, for example using a fast Fourier transform (FFT), and to further evaluate the resulting first frequency signal to ascertain the first α characteristic within the α frequency range and to ascertain the first β characteristic within the β frequency range. The second α characteristic and the second β characteristic of the at least one second partial measurement signal (for closed eyes) are ascertained analogously to the first α characteristic and first β characteristic. The control and evaluation unit is in particular also configured to transform the at least one second partial measurement signal (for closed eyes) into the frequency domain, for example likewise using a fast Fourier transform (FFT), and to further evaluate the resulting second frequency signal to ascertain the second α characteristic within the α frequency range and to ascertain the second β characteristic within the β frequency range. Further evaluation is performed for example by determining the areas formed under the curve of the first or second frequency signal within the α frequency range and within the β frequency range. However, the α and β characteristics may also be ascertained in another suitable manner, for example by determining the energy contents of the at least one first partial measurement signal (for open eyes) and the at least one second partial measurement signal (for closed eyes) within the α frequency range and within the β frequency range, the relevant signal additionally being squared.

To remove the influence of random fluctuations, the control and evaluation unit may in particular be configured to take an average over a plurality of individual measurements or individual evaluations.

In a further favourable embodiment, the control and evaluation unit is configured to determine the relative change between the first and the second α/β ratio as a difference, based on the first α/β ratio, between the first α/β ratio as the minuend and the second α/β ratio as the subtrahend, and to decide on the presence of moderate to severe insomnia if the relative change thus determined between the first and the second α/β ratio is greater than an insomnia threshold. The insomnia threshold is in particular in the range between −45% and −15%, preferably in the range between −35% and −17%, most preferably in the range between −25% and −19%, and preferably at −20%.

In a further favourable embodiment, the control and evaluation unit is configured to determine a degree of insomnia IG in accordance with the equation:

$$IG = {e}^{( \frac{Δ α β + C 1}{c 2} )} , \qquad ( 1 )$$

$$where$$

$$Δ αβ = \frac{( \frac{α1}{β1} - \frac{α2}{β2} )}{\frac{α1}{β1}} , \qquad ( 2 )$$

where c1 is a first constant, c2 a second constant and ΔαΔ the relative change between the first and the second α/β ratio. Furthermore, α1 represents the first α characteristic during the eyes-open period, β1 the first β characteristic during the eyes-open period, α2 the second α characteristic during the eyes-closed period and β2 the second β characteristic during the eyes-closed period. In a particularly preferred embodiment, the first constant c1 has a value of in particular 110.4 and the second constant c2 has a value of in particular 31.5. The degree of insomnia IG can thus be determined very reliably and precisely. The sensitivity of this determination is in particular 0.93 and the specificity is in particular 0.76.

In a further favourable embodiment, the control and evaluation unit is configured to carry out the objective assessment routine before the patient falls asleep, in particular immediately before he falls asleep. The objective assessment routine is preferably carried out before the usual or actual sleeping hours, for example before the evening bedtime. This is favourable because vigilance shortly before sleep or even at the onset of sleep is independent of the subsequent sleep pattern, and so better insomnia diagnosis outcomes can be achieved at this time.

In a further favourable embodiment, the device has at least one portable subcomponent. The portable subcomponent may in particular be a mobile device, such as a smartphone, a tablet computer, a laptop computer or a wearable. The device is preferably configured as a device operable by the patient himself, in particular in his home environment. Preferably, the device is deployable or usable in the patient's home environment. This facilitates handling and leads to very good results, since the patient does not have to leave his usual sleeping environment and undergo an examination for example in a sleep laboratory to diagnose insomnia. This situation can also lead to a distorted result, since sleeping behaviour can change as a result of the unfamiliar environment.

In a further favourable embodiment, the control and evaluation unit is configured to perform artefact removal on the EEG measurement signal before the at least one first partial measurement signal and the at least one second partial measurement signal are extracted. This improves the accuracy of the variables and parameters derived from the recorded EEG measurement signal, in particular those for insomnia diagnosis.

In a further favourable embodiment, the control and evaluation unit is configured to provide a duration of at least 2 seconds for each of the at least one eyes-open period and the at least one eyes-closed period. An upper bound on this duration is in particular in the range between 7 seconds and 25 seconds, preferably between 8 seconds and 20 seconds, and preferably at 10 seconds. The lower bound of 2 seconds ensures that the first and second partial measurement signals extracted from the EEG measurement signal are sufficiently long and predictive for the subsequent evaluation.

Shorter durations are also not very practical, as the patient often does not react quickly enough. The stated values (ranges) for the upper bound on the duration lead to predictive first and second partial measurement signals which can be evaluated well, and also do not require unreasonably great patience from the patient while he remains in the required eyelid position (eyes open or eyes closed).

In a further favourable embodiment, the control and evaluation unit is configured to provide at least two eyes-open periods and at least two eyes-closed periods during the objective assessment routine, the eyes-open periods and the eyes-closed periods alternating and in particular following one another directly. The more eyes-open periods and eyes-closed periods are stipulated during the objective assessment routine, the more or the better it is possible to extract analysable first and second partial measurement signals from the recorded EEG measurement signal. For example, two or three each of eyes-open periods and eyes-closed periods may be provided. A configuration having three eyes-open periods and two eyes-closed periods is preferred.

In a further favourable embodiment, while the objective assessment routine is being carried out, the control and evaluation unit is configured to indicate to the patient, in particular visually and/or acoustically, when the at least one eyes-open period and the at least one eyes-closed period each begin and end. The indication may in particular be purely acoustic or a mixture of visual and acoustic. In this way, the start of the eyes-closed period can be indicated visually or acoustically. The end of the eyes-closed period, on the other hand, can only reasonably be indicated by an acoustic indication, such as the playback of an announcement or even just a signal tone, since the patient will not recognise a visual indication if his eyes are closed. A request to start the eyes-closed period can be made by means of a visual indication, for example by means of on-screen text or an, in particular moving or shrinking, screen symbol. In this way, an initially relatively large dot shown on a display can become smaller and, when it disappears completely, provide the start signal to close the eyes. An in particular visual and/or acoustic indication of this type for the start and end of the eyes-open or eyes-closed period in question simplifies the handling of the device and also improves recording accuracy. Furthermore, it is in particular also possible to ascertain the eyelid position (eyes open or closed) from the progression of an EMG measurement signal being another, in particular also recorded, electrophysiological measurement signal which allows tracking of muscle activity in the eyelids, or to check whether the patient has followed the instruction to open or close his eyes.

In a further favourable embodiment, the device is configured with multiple components. In particular the control and evaluation unit may be configured in a plurality of parts or with multiple components and/or at least partially, preferably entirely, as a mobile or portable unit. It may have as subcomponents at least one, in particular portable, pre-processing unit, which can be placed for example near the electrodes used for recording measuring signals, for example on the patient's head, and a computing unit, in particular portable and placeable by the patient's bed, such as a tablet computer, a smartphone, a laptop computer or a wearable. There is a data or communication link, in particular a radio data or radio communication link, for example using the Bluetooth standard, between the individual subcomponents of the control and evaluation unit. The subdivision into a plurality of subcomponents simplifies the handling of the device and improves the flexibility of its use.

In a further favourable embodiment, the control and evaluation unit is configured to carry out a subjective assessment routine and in doing so to conduct a survey with the patient regarding his subjective sleep perception, in particular before he falls asleep in the evening or after he wakes up the next morning, and in particular to make a further insomnia determination on the basis of the survey results. In particular, the control and evaluation unit is configured to compare the results of the objective assessment routine and the results of the subjective assessment routine with one another or to refine the results of the objective assessment routine on the basis of the results of the subjective assessment routine. This increases the accuracy of the insomnia determination.

Further features, advantages, and details of the invention will become apparent from the following description of example embodiments with reference to the drawings, in which:

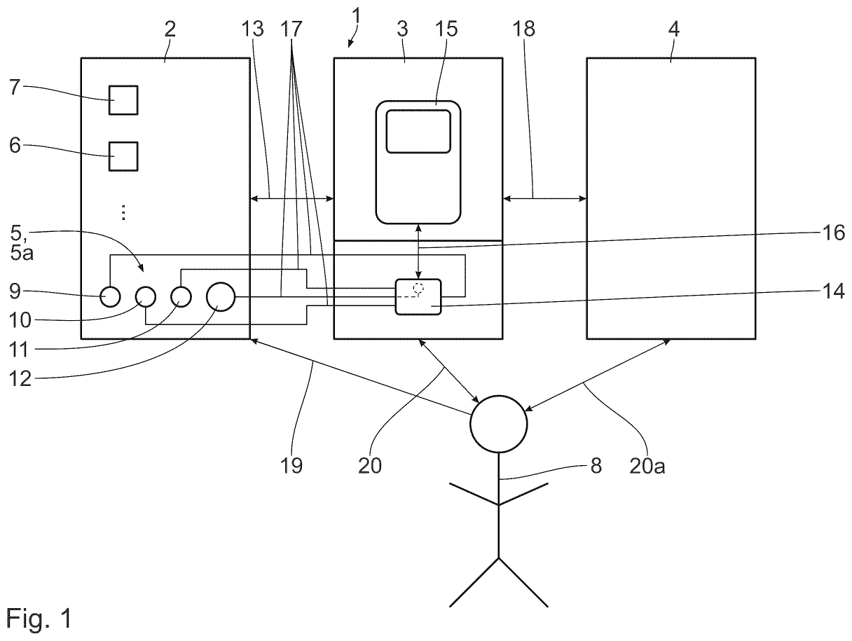

FIG. 1 is a block diagram of an example embodiment of an insomnia determination device, comprising a sensor unit and a control and evaluation unit,

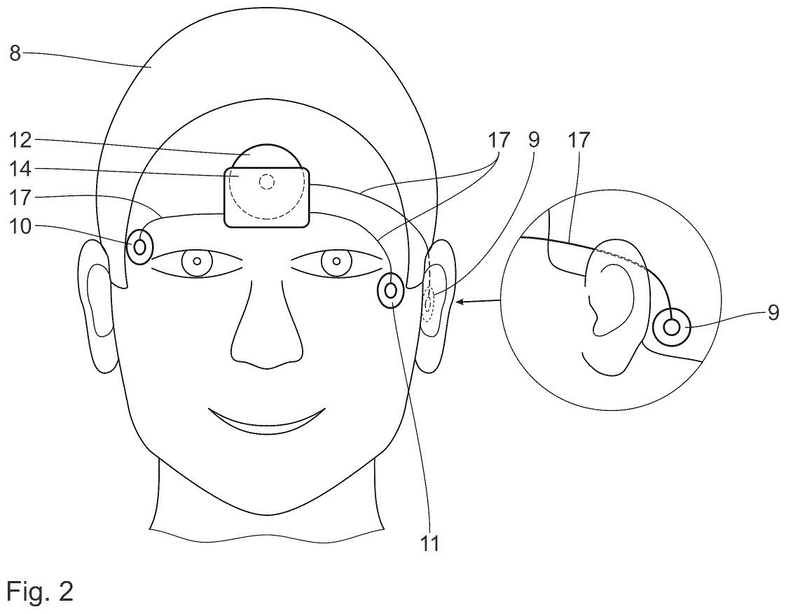

FIG. 2 is a partial representation of an example embodiment of an insomnia determination device according to FIG. 1, comprising measuring electrodes attached to the head of a patient for recording electrophysiological measuring signals as part of the sensor unit and a pre-processing unit as part of the control and evaluation unit,

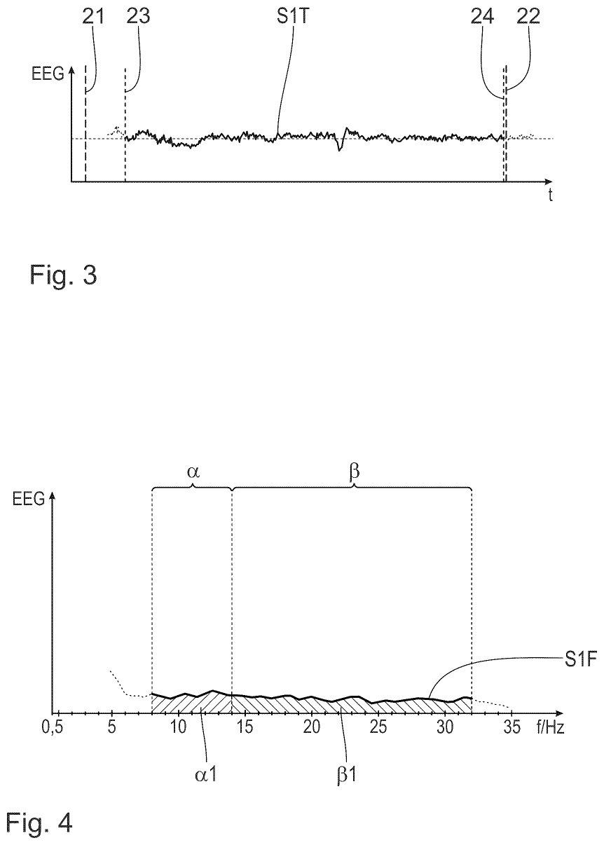

FIG. 3 is a graph over time of a first partial measurement signal of an EEG measurement signal recorded from a healthy individual, from an eyes-open period,

FIG. 4 is a frequency response graph of the first partial measurement signal of FIG. 3 transformed into the frequency domain,

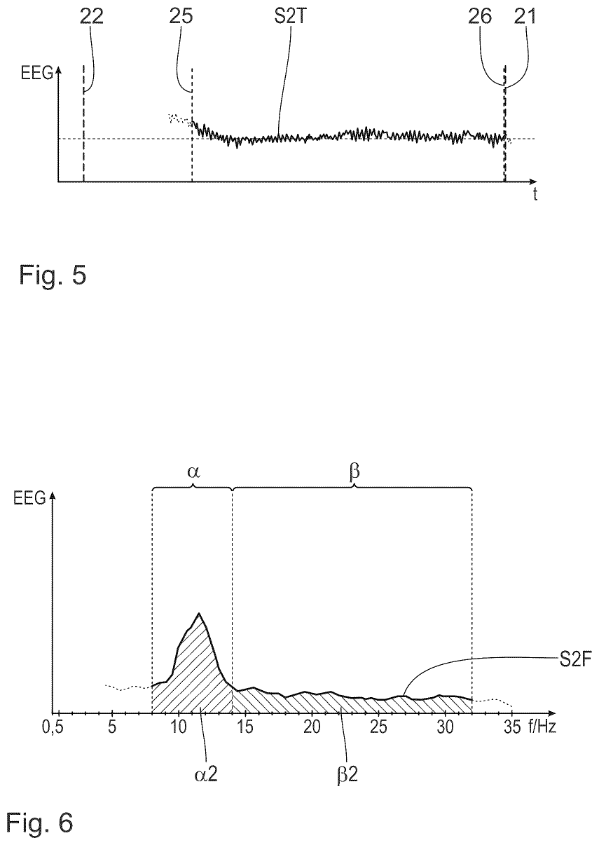

FIG. 5 is a graph over time of a second partial measurement signal of an EEG measurement signal recorded from a healthy individual, from an eyes-closed period,

FIG. 6 is a frequency response graph of the second partial measurement signal of FIG. 5 transformed into the frequency domain,

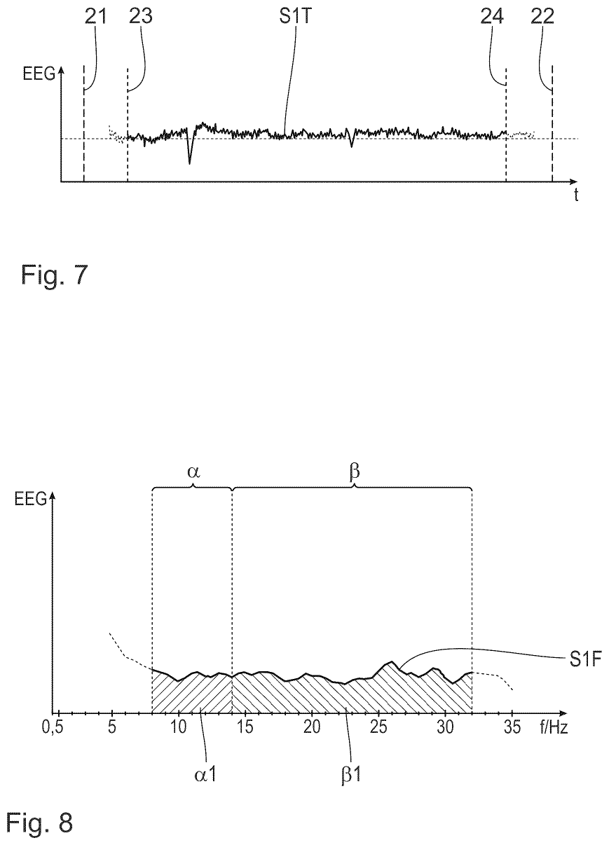

FIG. 7 is a graph over time of a first partial measurement signal of an EEG measurement signal recorded from an insomniac, from an eyes-open period,

FIG. 8 is a frequency response graph of the first partial measurement signal of FIG. 7 transformed into the frequency domain,

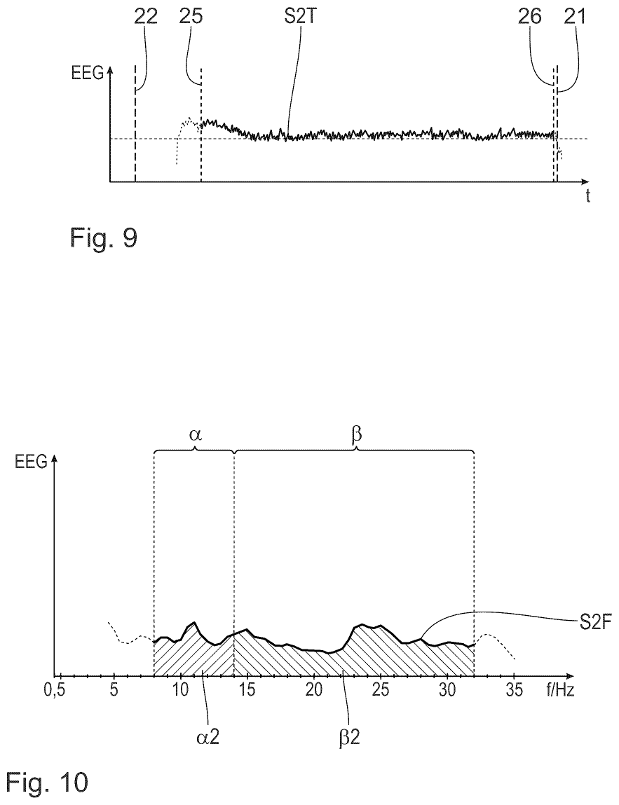

FIG. 9 is a graph over time of a second partial measurement signal of an EEG measurement signal recorded from an insomniac, from an eyes-closed period,

FIG. 10 is a frequency response graph of the second partial measurement signal of FIG. 9 transformed into the frequency domain,

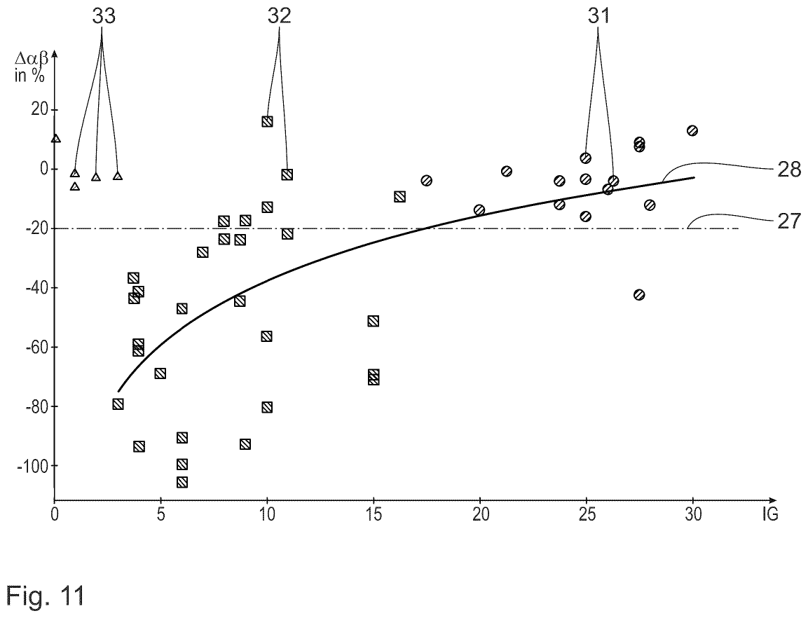

FIG. 11 is a graph showing the relative change between the first α/β ratio with eyes open and the second α/β ratio with eyes closed, plotted against the degree of insomnia,

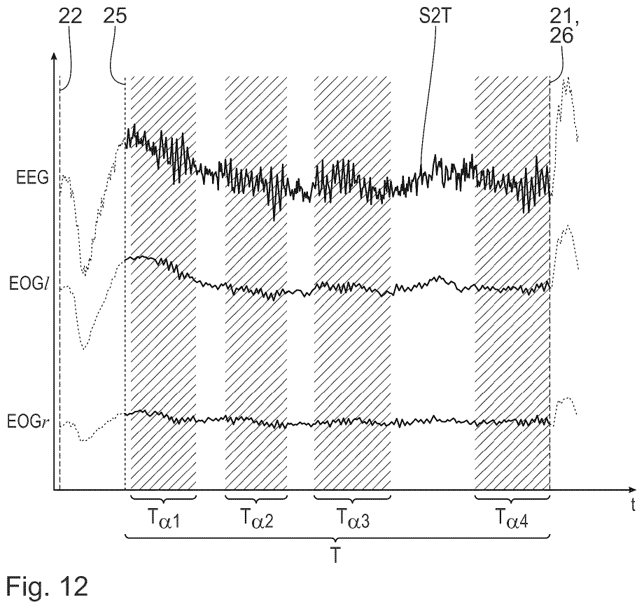

FIG. 12 is a graph over time of a second partial measurement signal of an EEG measurement signal recorded from a healthy individual, from an eyes-closed period, and of two EOG measurement signals, and

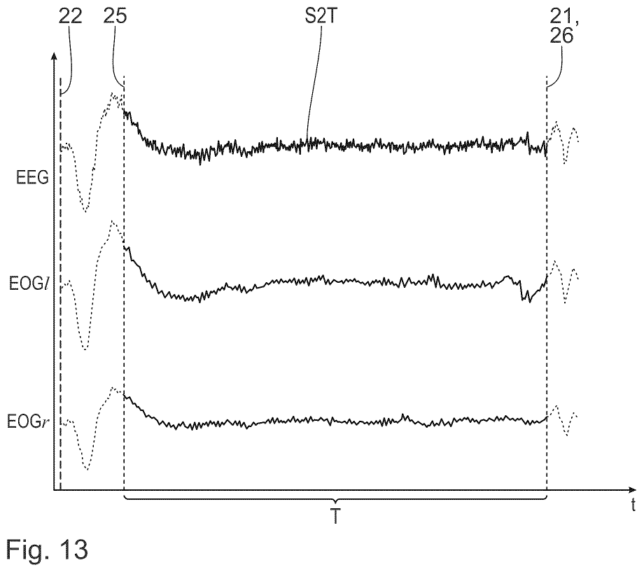

FIG. 13 is a graph over time of a second partial measurement signal of an EEG measurement signal recorded from an insomniac, from an eyes-closed period, and of two EOG measurement signals.

Corresponding parts are provided with like reference numerals in FIGS. 1 to 13. Details of the example embodiments explained in greater detail in the following may also constitute an invention in their own right or be part of a subject matter of the invention.

FIG. 1 shows an example embodiment of an insomnia determination device 1 in a block diagram. The insomnia determination device 1 comprises a sensor unit 2, a control and evaluation unit 3, and an optional remote unit 4.

The sensor unit 2 contains a plurality of sensors 5, 6 and 7, which are intended to record measurement signals related to the sleep behaviour of a patient 8. The sensors 5 are configured to record electrophysiological measurement signals and contain for this purpose a plurality of measuring electrodes 9, 10, 11 and 12, which are to be placed on the scalp of the patient 8. According to the drawing of FIG. 2, the measuring electrode 9, configured in particular as an earth electrode, is to be placed behind an ear of the patient 8. The measuring electrode 10 is to be placed to the right of the eyes, and the measuring electrode 11 is to be placed opposite, to the left of the eyes. The measuring electrode 12 is to be placed in particular in the centre of the forehead. The measuring electrodes 9 to 12 each record electrical potentials. The potential differences between two of the measuring electrodes 9 to 12 in each case are recorded as electrophysiological measurement signals. Thus, the potential difference between the central measuring electrode 12 and the earth electrode 9 provides an EEG (electroencephalography) measurement signal for recording the electrical activity of the brain, in other words brain waves, the potential difference between the left measuring electrode 11 and the central measuring electrode 12 provides an EOG (electrooculography) measurement signal for the movement of the left eye, the potential difference between the right measuring electrode 10 and the central measuring electrode 12 provides another EOG measurement signal for the movement of the right eye, and the potential difference between the right measuring electrode 10 and the left measuring electrode 11 provides an EMG (electromyography) measurement signal for recording muscle activity in this head region. The pair of the central measuring electrode 12 and the earth electrode 9 constitutes an EEG sensor 5a, at least the central measuring electrode 12 additionally being usable for other sensory recordings, in particular for recording EOG measurement signals. In addition, further sensors are present in the sensor unit 2, namely at least the sensors 6 and 7. The sensor 6 is configured as a position sensor and is used to record the head position and/or head movements of the patient 8. The sensor 7 is an acoustic sensor and is used to record snoring noises of the patient 8. Further sensors may be present, for example a light sensor for recording ambient light. The measurement signals recorded by the sensors 5 to 7 are transmitted to the control and evaluation unit 3. For this purpose, a unidirectional or bidirectional communication link 13 is available, which is configured either as a wired communication link 17 or as a wireless communication link, for example using the Bluetooth standard. In the example embodiment shown, the communication between the sensors 5 or their measuring electrodes 9 to 12 and the control and evaluation unit 3 is wired, and the communication between the further sensors 6 and 7 and the control and evaluation unit 3 is wireless. However, other subdivisions or variants are also possible in principle.

In the example embodiment shown, the control and evaluation unit 3 is configured in multiple parts or multiple components. It comprises a pre-processing unit 14 and a mobile computing unit 15 in the form of a tablet computer. Between the pre-processing unit 14 and the mobile computing unit 15, there is also a communication link 16, which in the example embodiment shown is preferably implemented as a radio communication link using the Bluetooth standard. It is in particular bidirectional. As shown in FIG. 2, the pre-processing unit 14 is exposed to the central measuring electrode 12, an electrical communication link simultaneously being established between these two components. There is a wired communication link 17 for each of the remaining measuring electrodes 9, 10, and 11.

The remote unit 4 is configured as a stationary computing unit located remotely from the patient 8, for example as a server or a cloud computer. The remote unit 4 may also have a data storage device with a large storage volume, such as a cloud storage device. For data exchange, there is an Internet communication link 18 between the remote unit 4 and the control and evaluation unit 3. The data ascertained by the sensor unit 2 and/or the control and evaluation unit 3 can be transmitted to the remote unit 4, for example so as to perform further evaluations there or to offer a medical professional access for his assessment.

The patient 8 interacts with the various units of the insomnia determination device 1. In the case of the sensor unit 2, this interaction relates to physical variables which can be recorded from the patient by the sensors 5 to 7 of the sensor unit 2. This interaction 19 is thus directed from the patient 8 to the sensor unit 2. It is in particular unidirectional. The interaction 20 between the patient 8 and the control and evaluation unit 3, by contrast, is configured bidirectional, as is the interaction 20a between the patient 8 and the remote unit 4. The patient 8 can receive information from the control and evaluation unit 3, for example visually or acoustically. Conversely, the patient 8 can provide inputs to the control and evaluation unit 3, for example as part of an initial calibration routine, an objective assessment routine and/or a subjective assessment routine involving a survey of the patient 8.

During the calibration routine, the basic settings of the insomnia determination device 1 are ascertained, in particular from the conditions currently prevailing in the patient 8. Furthermore, the position and fit of the measuring electrodes 9 to 12 applied by the patient 8 are checked, in particular by means of a photograph.

The insomnia determination device 1 is distinguished in that it can be operated by the patient 8 himself and, above all, can also be used in his home environment.

The control and evaluation unit 3 is configured to carry out an objective assessment routine in which stipulations regarding eye opening and eye closure are given to the patient 8 so as also to cover periods during the recording of the EEG measurement signal where the eyes of the patient 8 are open (=eyes-open periods) and closed (=eyes-closed periods). In the subsequent evaluation of the EEG measurement signal thus recorded, partial measurement signals are extracted which fall precisely within specific periods of this type. First partial measurement signals S1T each fall within an eyes-open period, and second partial measurement signals S2T each fall within an eyes-closed period. First partial measurement signals S1T are shown by way of example in the graph of FIG. 3 (for a healthy individual) and in FIG. 7 (for an individual suffering from insomnia (=insomniac)).

Analogously, second partial measurement signals S2T are shown as examples in FIG. 5 (for a healthy individual) and in FIG. 9 (for an insomniac). These graphs each show curves plotted against time t. The time curves according to FIGS. 3, 5, 7 and 9 include the times 21 at which the control and evaluation unit 3 issued a request to open the eyes and the times 22 at which the control and evaluation unit 3 issued a request to close the eyes. The beginning of the extracted first partial measurement signal S1T is labelled as 23 and its end as 24, and the beginning of the extracted second partial measurement signal S2T is labelled as 25 and its end as 26. The duration of the first and second partial measurement signals S1T, S2T is shorter in each case than the eyes-open and eyes-closed periods stipulated by the control and evaluation unit 3, since, when the eyelid position changes, transition effects occur in the recorded EEG measurement signal, which would distort the further evaluation and are therefore not taken into account. The control and evaluation unit 3 is also configured to eliminate other artefacts so as to increase the quality of the subsequent evaluation. This artefact removal may in particular also take place even before the extraction of the first and second partial measurement signals S1T, S2T. The duration of the extracted first and second partial measurement signals S1T, S2T is at least 2 seconds and is typically in the range of 5 to 10 seconds.

The extracted first and second partial measurement signals S1T, S2T are transformed into the frequency domain using a fast Fourier transform (FFT), resulting in the respectively associated transformed first frequency signals S1F (for open eyes) and second frequency signals S2F (for closed eyes). The first frequency signals SIF thus formed are shown in the frequency graphs of FIGS. 4 and 8, and the second frequency signals S2F in the frequency graphs of FIGS. 6 and 10. The first and second frequency signals S1F, S2F are each plotted against the frequency f. Of these frequency responses, only the α frequency range between 8 Hz and 14 Hz and the adjacent β frequency range between 14 Hz and 32 Hz are of interest here. The α and β frequency ranges are identified in FIGS. 4, 6, 8, and 10. The EEG measurement signal records the brainwave activity of patient 8. This activity changes. The EEG measurement signal can therefore also contain different components, each having a different frequency content. In particular, it may include what are known as α waves, whose frequencies fall in the aforementioned α frequency range and which usually occur when the patient 8 is awake and has his eyes closed. Furthermore, the EEG measurement signal may also include what are known as β waves, whose frequencies fall in the likewise aforementioned β frequency range and which usually occur when the patient 8 is awake and has his eyes open. If the patient 8 changes the position of his eyelids, this can also be read off in the frequency response in healthy individuals. What is significant here is the comparison of the first frequency signal SIF shown in FIG. 4 with the second frequency signal S2F shown in FIG. 6. It is noticeable that the frequency content in the α frequency range is higher for the second frequency signal S2F (eyes closed) of FIG. 6 than for the first frequency signal S1F (eyes open) of FIG. 4. This difference is typical and is referred to as the Berger effect. It has been found that this change in the frequency content within the α frequency range upon changing between open eyes and closed eyes does not occur in insomniacs in a comparable manner to in healthy individuals. This can also be seen from the relevant graphs in FIGS. 8 and 10. In insomniacs, there is no increase in the frequency content in the α frequency range when eyes are closed as in healthy individuals. This finding is used in the insomnia determination device 1 for an objective determination of insomnia or of a degree of insomnia.

Thus, the control and evaluation unit 3 is configured to ascertain first α characteristics α1 and first β characteristics β1 from the first frequency signal S1F, namely in particular by determining the area under the curve of the frequency signal SIF within the α frequency range and within the β frequency range. The relevant areas for the first α characteristic α1 and the first β characteristic β1 are identified by different hatching in the graphs of FIGS. 4 and 8. Accordingly, a second α characteristic α2 and a second β characteristic β2 are ascertained using the second frequency signal S2F. In this case too, areas below the curves of the second frequency signal S2F are ascertained. The corresponding areas are again identified by hatching in the relevant FIGS. 6 and 10. From these α and β characteristics α1, α2, β1, β2, the control and evaluation unit takes a first α/β ratio, as the quotient of the first α characteristic α1 over the first β characteristic β1, and a second α/β ratio, as the quotient of the second α characteristic α2 over the second β characteristic β2. From this, the relative change ΔαΔ between the first and the second α/β ratio is found in accordance with equation (2) above.

In healthy individuals, there is a significant change between the first α/β ratio with eyes open and the second α/β ratio with eyes closed, whereas in insomniacs this relative change either does not occur at all or is very small. The relative change ΔαΔ between the first and the second α/β ratio can thus be used as a measure of insomnia in the patient 8.

This is an objective determination of insomnia, leading to advantages over the previously exclusively used questionnaire method, which is heavily determined by the subjective perceptions of the patient 8. Using equation (1) above, the degree of insomnia IG of the patient 8 can be determined from the relative changes ΔαΔ, ascertained by measurement and subsequent evaluations, between the first α/β ratio with eyes open and the second α/β ratio with eyes closed. The first constant c1 is in particular 110.4, and the second constant c2 is in particular 31.5. The functional relationship according to equation (1) or its inverse formulation according to the following equation (3):

$$Δ a β = [ c 2 * ln ( IG ) ] - c 1 \qquad ( 3 )$$

is represented in the graph of FIG. 11 by the curve 28 shown in solid lines. As a result of this functional relationship, the insomnia determination device 1 is able to indicate a degree of insomnia IG for the patient 8 who has undergone the objective assessment routine. This objective assessment routine is preferably carried out before falling asleep, and this also leads to advantages over the questionnaire method, which is only carried out after the sleep phase the following morning under the potentially misleading impressions of the previous sleep phase.

As an alternative or in addition to the functional determination of a degree of insomnia IG, the control and evaluation unit 3 may also simply check whether the ascertained relative change ΔαΔ between the first and the second α/β ratio exceeds an insomnia threshold 27 (represented as a dot-dash horizontal line in the graph of FIG. 11). In the example embodiment shown, the insomnia threshold 27 is −20%. If the relative change ΔαΔ exceeds this value, the control and evaluation unit 3 decides that moderate to severe insomnia is present.

The graph in FIG. 11 also includes the results of a series of tests. The test subjects underwent both the questionnaire method for insomnia diagnosis and the objective assessment routine of the insomnia determination device 1, so that for each of them there is a degree of insomnia IG ascertained in accordance with the questionnaire method and a relative change Δαβ between the first and the second α/β ratio ascertained in accordance with the insomnia determination device 1. The two results are assigned to one another as the outcome for the associated test subject and entered in the graph of FIG. 11. The hatched circles 31 refer to the results of insomniacs, the hatched squares 32 to the results of healthy subjects, and the hatched triangles 33 to the results of test subjects for whom measurement errors were recorded and who are therefore to be considered outliers. These results from the test subjects were subjected to a logarithmic fit, resulting in the functional relationship according to equations (1) and (3), shown in FIG. 11 by the curve 28. There is a very high sensitivity of 0.93 and a likewise very high specificity of 0.76.

The control and evaluation unit 3 may in particular be configured to carry out a subjective assessment routine based on the questionnaire method in addition to the described objective assessment routine. The results of this subjective assessment routine can then be used to verify and/or improve the results of the objective assessment routine.

In the following, an alternative method for objective insomnia determination, which may additionally or alternatively be implemented in the insomnia determination device 1, is described with reference to FIGS. 12 and 13.

FIGS. 12 and 13 each show three graphs over time of an EEG measurement signal, an EOG measurement signal of the left eye (labelled EOGl in FIGS. 12 and 13) and an EOG measurement signal of the right eye (labelled EOGr in FIGS. 12 and 13). The measurement signals of FIG. 12 come from a healthy individual, whilst those of FIG. 13 come from an insomniac. In each case, the eyes-closed period is shown, during which a discernible and significant proportion of α waves is present in the healthy individual, whilst a comparable proportion of α waves is not present in the insomniac individual.

Of the EEG measurement signals of FIGS. 12 and 13, again only the second partial measurement signals S2T, which have been cleaned of artefacts, are analysed in greater detail. The disregarded artefacts occur primarily (but not exclusively) at the start between the associated time 22 of the request to close the eyes and the associated beginning 25 of the second partial measurement signal S2T which is further analysed. The movements of the eyelids due to the request to close the eyes, discernible from the EOG measurement signals of FIGS. 12 and 13, occur in this phase. The end 26 of the second partial measurement signal S2T and the time 21 of the request to open the eyes coincide in the time curves shown by way of example in FIGS. 12 and 13.

The control and evaluation unit 3 is configured to analyse the second partial measurement signals S2T as to whether they have a periods in which α waves are predominant, in particular significantly so, in particular over other wave components of the second partial measurement signal S2T and/or are dominant in particular by comparison with other wave components of the second partial measurement signal S2T. This is done for example using pattern recognition. The second partial measurement signal S2T of FIG. 12, recorded from the healthy individual, has 4 α periods of this type having a period durations T_{α1}, T_{α2}, T_{α3} and T_{α4}. By contrast, the second partial measurement signal S2T of FIG. 13 recorded from the insomniac has no a period. Both situations are detected by the control and evaluation unit 3 and used for the derived insomnia diagnosis. Accordingly, the presence of moderate to severe insomnia is decided if the condition of the following equation (4):

$$\frac{{\sum }_{i} {T}_{α i}}{T} < 0.2 \qquad ( 4 )$$

is met, where T is the total duration of the respectively analysed second partial measurement signal S2T, T_{αi} is the α period duration of a recorded α period in which α waves are in particular dominant and/or preferably significantly predominant, and i is a running index.

In the example of FIG. 12, the α period durations have the following values in seconds: T_{α1}=1.1 s, T_{α2}=1.0 s, T_{α3}=1.2 s, and T_{α4}=1.2 s, and the total duration of the analysed second partial measurement signal S2T is T=7 s. Equation (4) gives a value of 0.64, which is greater than the threshold value of 0.2, and so the control and evaluation unit 3 does not record insomnia in this case.

In the example of FIG. 13, the total duration of the analysed second partial measurement signal S2T is T=8 s. However, there are no recorded α periods, so the sum of all α period durations T_{αi} gives a value of zero. Equation (4) therefore also gives a value of zero, which is less than the threshold of 0.2, and so the control and evaluation unit 3 decides that insomnia is present in this case.

Overall, the insomnia determination device 1 provides, for the first time, the possibility of determining insomnia objectively. The insomnia determination device 1 can advantageously be operated by the patient 8 himself and, above all, can be used in his home environment, and this eliminates misdiagnoses due to an unfamiliar sleeping environment and is considerably more convenient for the patient 8.

1. A device for determining insomnia in a patient, comprising
a) a sensor unit, at least for recording an EEG measurement signal of a brain wave of the patient, and
b) a control and evaluation unit for evaluating the recorded EEG measurement signal,
wherein
c) the control and evaluation unit is configured
c1) to carry out an objective assessment routine and in doing so,
c11) for the subsequent recording of the EEG measurement signal, to give the patient (8) a stipulation to open his eyes for the duration of an eyes-open period and to close his eyes for the duration of an eyes-closed period, and
c12) then to initiate the recording of the EEG measurement signal during at least one eyes-open period and during at least one eyes-closed period,
c2) to perform an evaluation of the EEG measurement signal thus recorded and in doing so
c21) to extract from the EEG measurement signal at least one first partial measurement signal from the at least one eyes-open period and at least one second partial measurement signal from the at least one eyes-closed period, and
c22) to decide on the presence of insomnia if the at least one second partial measurement signal from the at least one eyes-closed period has a lower proportion of α waves than in a healthy individual.

2. The device according to claim 1, wherein the control and evaluation unit is configured to check the at least one second partial measurement signal for the presence of a periods in which α waves occur, and to ascertain an α period duration for each recorded α period, and to decide on the presence of moderate to severe insomnia if the sum of all α period durations is less than 20% of a total period of the at least one second partial measurement signal.

3. The device according to claim 1, wherein the control and evaluation unit is configured to perform a comparison between a first frequency content of the at least one first partial measurement signal from the at least one eyes-open period and a second frequency content of the at least one second partial measurement signal from the at least one eyes-closed period, and, for the comparison,
a) to determine, from the first frequency content of the at least one first partial measurement signal from the at least one eyes-open period, a first α characteristic for an α frequency range and a first β characteristic for a β frequency range as well as a first α/β ratio as the quotient of the first α characteristic over the first β characteristic,
b) to determine, from the second frequency content of the at least one second partial measurement signal from the at least one eyes-closed period, a second α characteristic for the α frequency range and a second β characteristic for the β frequency range as well as a second α/β ratio as the quotient of the second α characteristic over the second β characteristic, and
c) to determine a relative change between the first and the second α/β ratio.

4. The device according to claim 3, wherein the control and evaluation unit is configured
(a) to determine the relative change between the first and the second α/β ratio as a difference, based on the first α/β ratio, between the first α/β ratio as the minuend and the second α/β ratio as the subtrahend, and
b) to decide on the presence of moderate to severe insomnia if the relative change thus determined between the first and the second α/β ratio is greater than an insomnia threshold.

5. Device according to claim 3, wherein the control and evaluation unit is configured to determine a degree of insomnia IG according to the equation:
IG=e^{(c2/Δαβ+c1)}
where
Δαβ=β1/α1/(β1/α1−β2/α2),
where c1 is a first constant, c2 a second constant, ΔαΔ the relative change between the first and the second α/β ratio, α1 the first α characteristic during the eyes-open period, β1 the first β characteristic during the eyes-open period, α2 the second α characteristic during the eyes-closed period and β2 the second β characteristic during the eyes-closed period.

6. The device according to claim 1, wherein the control and evaluation unit is configured to carry out the objective assessment routine before the patient falls asleep.

7. The device according to claim 1, wherein the device has at least one portable subcomponent.

8. The device according to claim 1, wherein the device is configured as a device operable by the patient himself.

9. The device according to claim 1, wherein the device is configured as a device operable by the patient in his home environment.

10. The device according to claim 1, wherein the control and evaluation unit is configured to perform artefact removal on the EEG measurement signal before the at least one first partial measurement signal and the at least one second partial measurement signal are extracted.

11. The device according to claim 1, wherein the control and evaluation unit is configured to provide a duration of at least 2 seconds for each of the at least one eyes-open period and the at least one eyes-closed period.

12. The device according to claim 1, wherein the control and evaluation unit is configured to provide at least two eyes-open periods and at least two eyes-closed periods during the objective assessment routine, the eyes-open periods and the eyes-closed periods alternating and in particular following one another directly.

13. The device according to claim 1, wherein, while the objective assessment routine is being carried out, the control and evaluation unit is configured to indicate to the patient when the at least one eyes-open period and the at least one eyes-closed period each begin and end.

14. The device according to claim 1, wherein it is configured as a multi-component device.

15. The device according to claim 1, wherein the control and evaluation unit is configured to carry out a subjective assessment routine and in doing so to conduct a survey with the patient regarding his subjective sleep perception and in particular to make a further insomnia determination based on the survey results.
